(19) United States
(12) Patent Application Publication
(10) Pub. No.: US 2018/0325617 A1
(43) Pub. Date: Nov. 15, 2018

(54) SURGICAL LIGHT AND USES THEREOF

(71) Applicant: MezLight, LLC, Madison, MI (US)

(72) Inventors: Joshua David Mezrich, Madison, WI (US); Craig James Christianson, Madison, WI (US); Christopher Charles Beglinger, Madison, WI (US)

(21) Appl. No.: 15/978,954

(22) Filed: May 14, 2018

Related U.S. Application Data
(60) Provisional application No. 62/505,595, filed on May 12, 2017.

(51) Int. Cl. A61B 90/30

(52) U.S. Cl. CPC A61B 90/30; A61B 1/0638; A61B 2090/308

(57) ABSTRACT

Provided herein are surgical lighting devices, systems, and methods. In particular, provided herein are sterile, disposable surgical lights and uses thereof.

### CROSS REFERENCE TO RELATED APPLICATIONS

The present invention claims the priority benefit of U.S. Provisional Patent Application 62/505,595, filed May 12, 2017 which is incorporated by reference in its entirety.

### FIELD

Provided herein are surgical lighting devices, systems, and methods. In particular, provided herein are sterile, disposable surgical lights and uses thereof.

### BACKGROUND

Surgeons typically utilize head lamps for providing direct light during surgery. These lamps require the user to maintain an uncomfortable and static head position in order to maintain uniform lighting of the surgical field. In addition, headlamps are not typically sterile and cannot be contacted by the surgeon to adjust aim or for comfort.

Even with these significant drawbacks, head lamps are commonly used because other existing lighting systems that are mounted above the patient, on the bed frame, or elsewhere in the surgical landscape are considered ineffective in terms of intruding into the work space and/or providing insufficient, adjustable, targetable light.

Improved devices and methods for surgical lighting are needed.

### SUMMARY

Provided herein are surgical lighting devices, systems, and methods. In particular, provided herein are sterile, disposable surgical lights and uses thereof.

The lighting devices, systems, and methods described herein solve many problems of the existing devices. Providing sterile devices allows users to manipulate the placement and aim of lights during procedures. This allows for better lighting of the procedure and improved comfort for the user.

For example, in some embodiments, the present disclosure provides a disposable, sterile, medical (e.g., surgical) light, comprising: a lamp operably linked to a multi-segment flexible arm comprising at least one rigid section and at least one flexible section. In some embodiments, the flexible arm comprises two flexible sections disposed on either side of a single rigid section. In some embodiments, the lengths of the flexible sections and the rigid section are present at a ratio of 1:2 to 2:1. In some embodiments, the light further comprises a power source (e.g., battery). In some embodiments, the light further comprises a support attachment component. In some embodiments, the support attachment component provides a component of the power source. In some embodiments, the light further comprises a camera. In some embodiments, the camera is attached to the flexible arm via a camera attachment clip. In some embodiments, the lamp is focusable. In some embodiments, the light further comprises an on-off switch. In some embodiments, the on-off switch is located on the power supply. In some embodiments, the lamp comprises at least one light source. In some embodiments, the lamp comprises at least two light sources, wherein each of the light sources emits light of a different wavelength or range of wavelengths. In some embodiments, the light source is configured to emit pulses of light. In some embodiments, the light source is configured to switch between the two light sources (e.g., as a rapid pulse).

Further embodiments provide a system, comprising: the light described herein; and a solid support configured to attach to the light using the support attachment component. In some embodiments, the solid support is sterilizable (e.g., via autoclave). In some embodiments, the solid support attaches to a medical procedure surface. In some embodiments, the solid support is attaches to the medical procedure surface using a clamp. In some embodiments, the medical procedure surface is a surgical bed (e.g., bed rail). In some embodiments, the height of the solid support is adjustable (e.g., via the clamp).

Yet other embodiments provide a method of lighting a medical procedure, comprising: a) contacting the system described herein with a medical procedure surface; and b) illuminating the medical procedure surface with the light. In some embodiments, the method further comprises the step of performing a medical procedure (e.g., surgery such as transplant surgery). In some embodiments, the position of the light is adjusted one or more times during the surgical procedure.

Additional embodiments are described herein.

### BRIEF DESCRIPTION OF THE DRAWINGS

FIG. 1 shows an image of an exemplary device of embodiments of the present disclosure.

FIG. 2 shows a close-up image of an exemplary device of embodiments of the present disclosure showing a camera component.

FIG. 3A-C shows an image of exemplary devices of embodiments of the present disclosure in use.

FIG. 4 shows a schematic of a lamphead assembly of an exemplary device of embodiments of the present disclosure.

FIG. 5 shows a schematic of a lamphead assembly of an exemplary device of embodiments of the present disclosure.

FIG. 6A-E shows a) a detail view of a flexible arm with battery connector; b) an overview of a flexible arm with battery connector; c) a flexible arm support; d) a flexible arm tube; and e) a flexible arm battery connector of an exemplary device of embodiments of the present disclosure.

FIG. 7 shows an overview of a battery component of an exemplary device of embodiments of the present disclosure.

FIG. 8 shows a balljoint cap of an exemplary device of embodiments of the present disclosure.

FIG. 9 shows a balljoint socket of an exemplary device of embodiments of the present disclosure.

FIG. 10A-C shows a) a left side view of a battery component; b) a right side view of a battery component; and c) a battery support of an exemplary device of embodiments of the present disclosure.

FIG. 11 shows an overview of the outside of a flexible arm with battery of an exemplary device of embodiments of the present disclosure.

FIG. 12A-E shows a) a lens; b) a lens retainer; c) a lamphead balljoint body; d) an aperture ring; and e) lamphead reflector of an exemplary device of embodiments of the present disclosure.

FIG. 13 shows a retainer ring of an exemplary device of embodiments of the present disclosure.

FIG. 14 shows a side lock of an exemplary device of embodiments of the present disclosure.

FIG. 15 shows a SMT of an exemplary device of embodiments of the present disclosure.

### DETAILED DESCRIPTION

Provided herein are surgical lighting devices, systems, and methods. In particular, provided herein are sterile, disposable surgical lights and uses thereof.

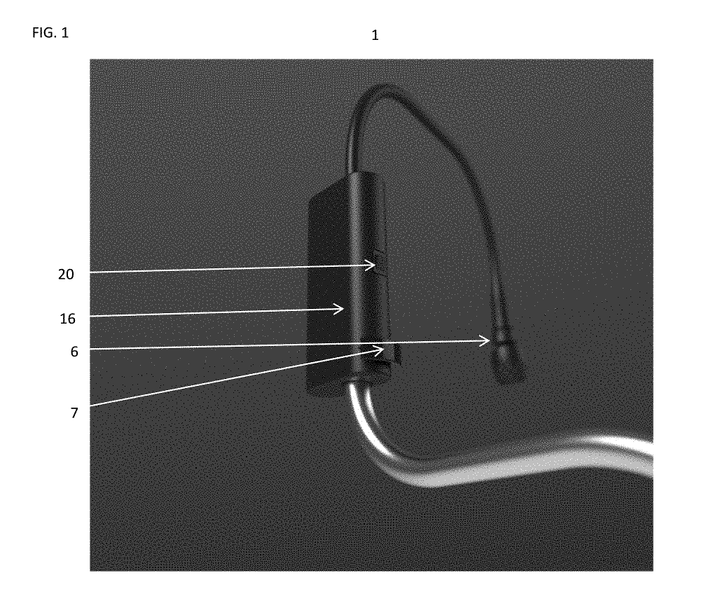

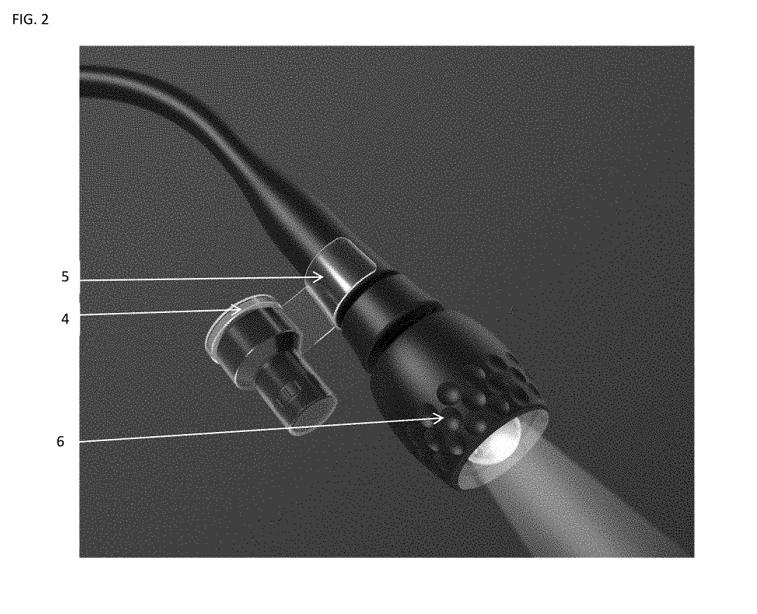

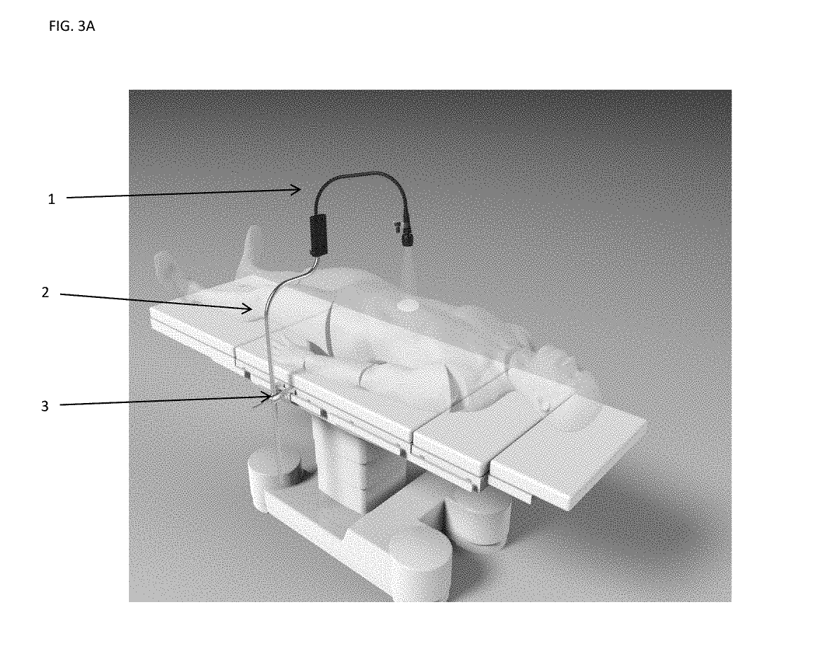

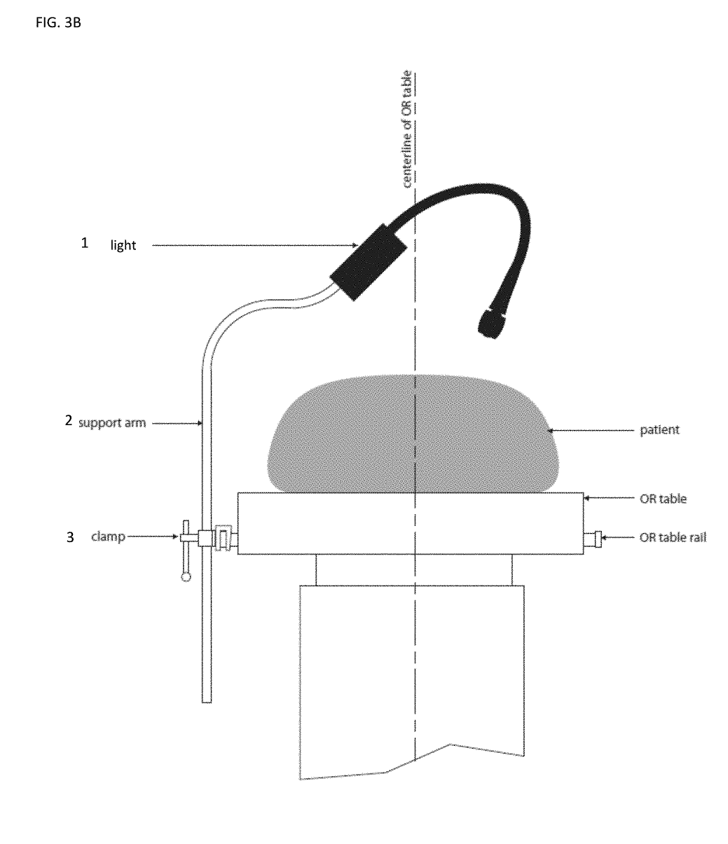

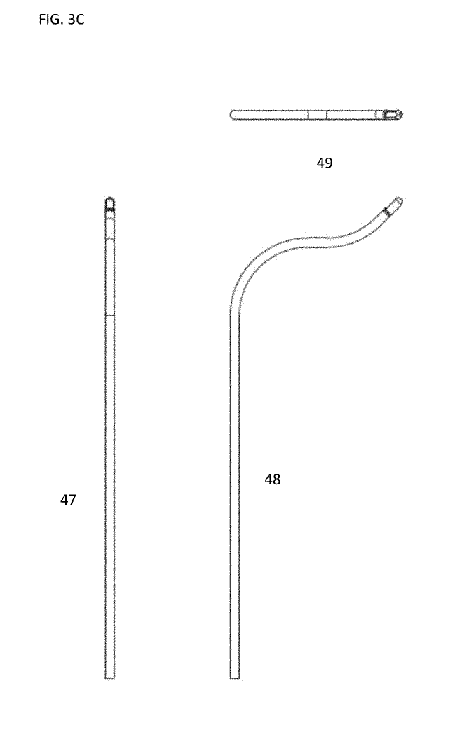

FIGS. 1-15 show exemplary devices and systems of the present disclosure. FIG. 1 shows a photograph of light 1 with lamp 6, power supply 16 with switch 20 and support attachment component 7. FIG. 2 shows a close-up image of an optional camera component 4 attached to light 1 with camera attachment clip 5. In some embodiments, camera 4 further comprises a microphone and or sound recording components (not shown). In some embodiments, a microphone is provided separately from the video camera. FIG. 3 shows an overview of the light 1 in use. Shown is light 1 attached to solid support 2. The solid support 2 is attached to a medical bed using clamp 3. FIG. 3B shows an alternative embodiment where the geometry of the support 2 is configured to aid in aligning the light 1 over the center line of the medical bed or operating table. In some embodiments, the support arm has a first, most distal segment that connects to the table or table rail, a second segment that runs approximately parallel to the plane of the table, and a third, most proximal segment, that attaches to the light component. In this embodiments, the third segment of the support arm is angled between 90° and 180° (preferably between 120° and 150°; e.g., 135°) relative to the second segment. FIG. 3C shows details of the support 2 shown in FIG. 3B, showing a back perspective 47, a side perspective 48, and a top perspective 49. In some embodiments, the support 2 comprises one or more straight or curved segments that lock together to form support 2.

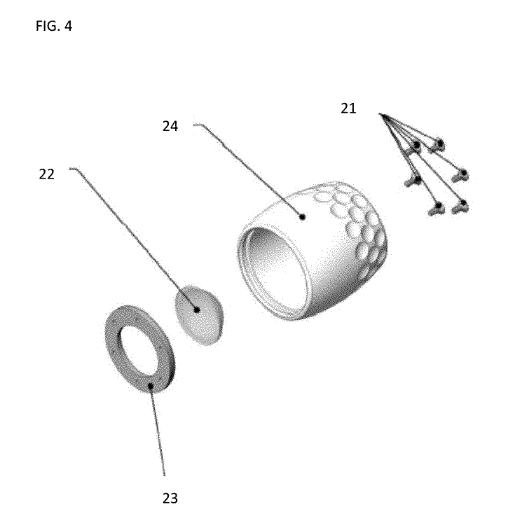

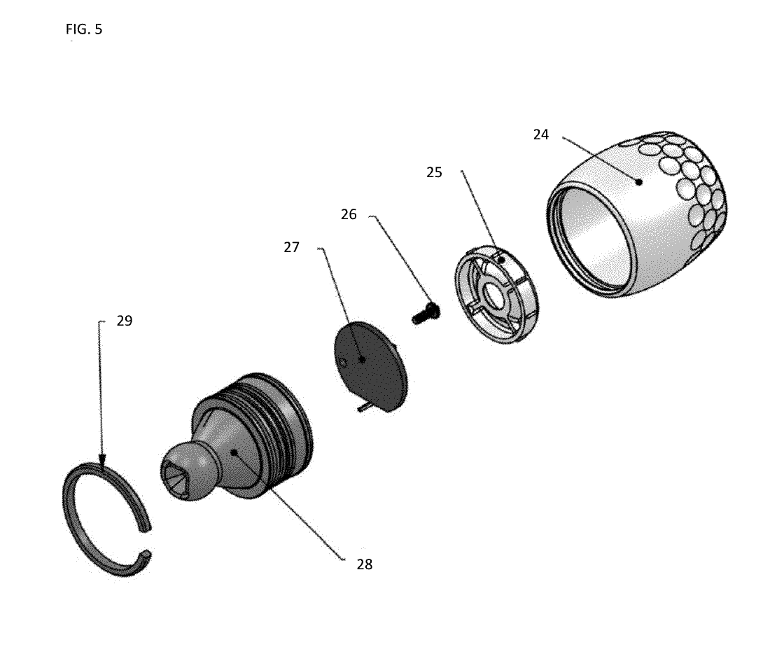

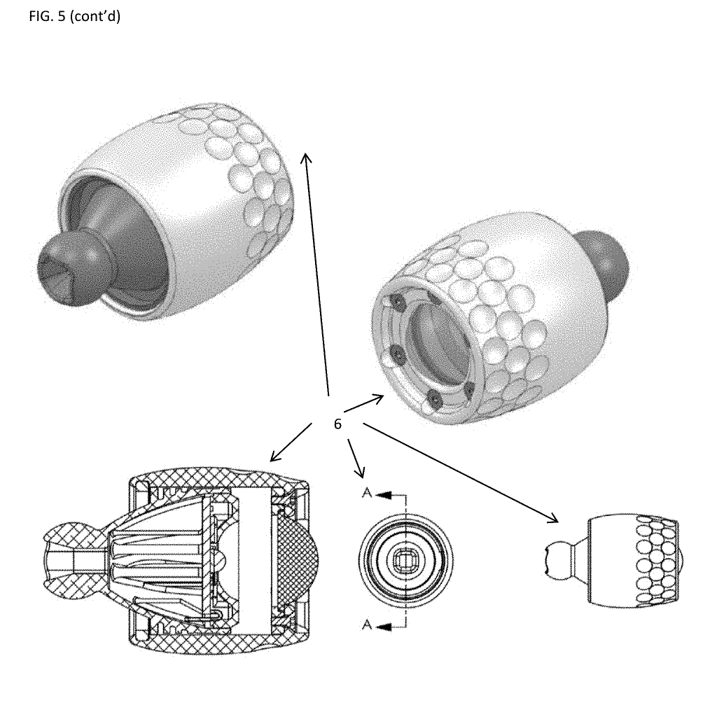

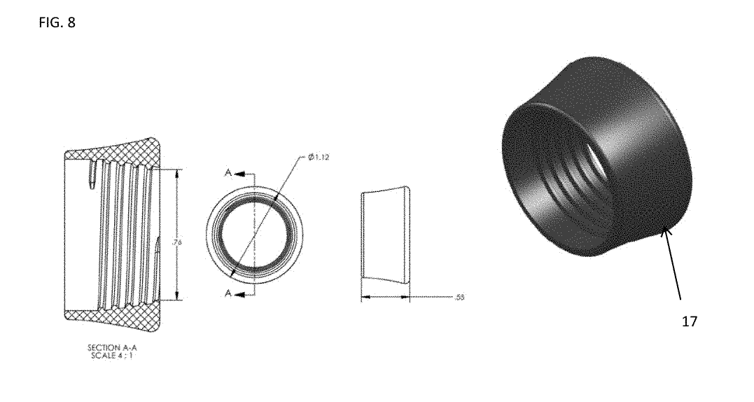

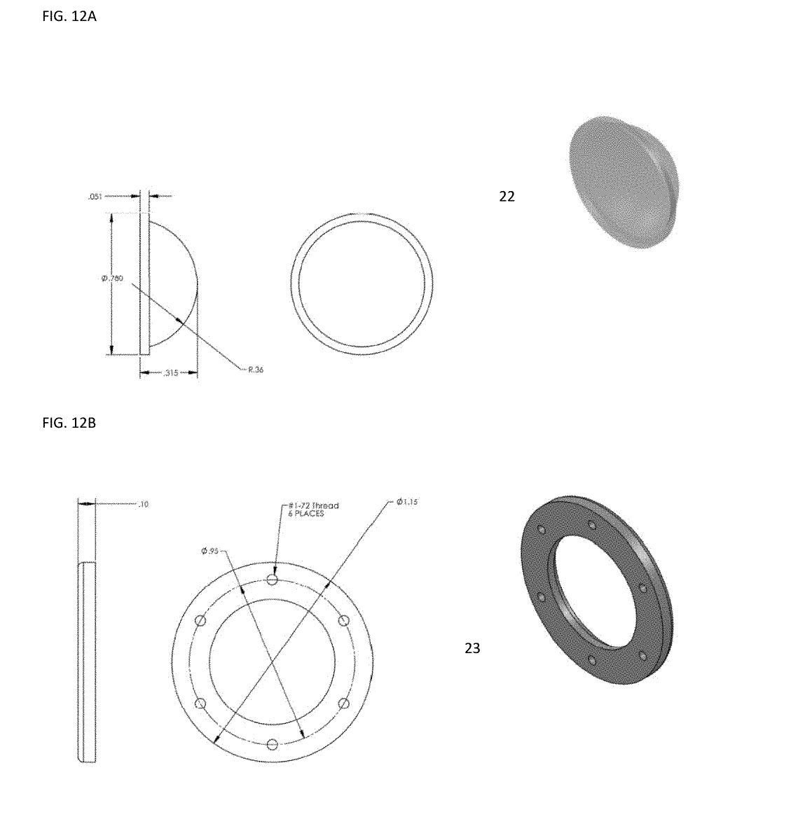

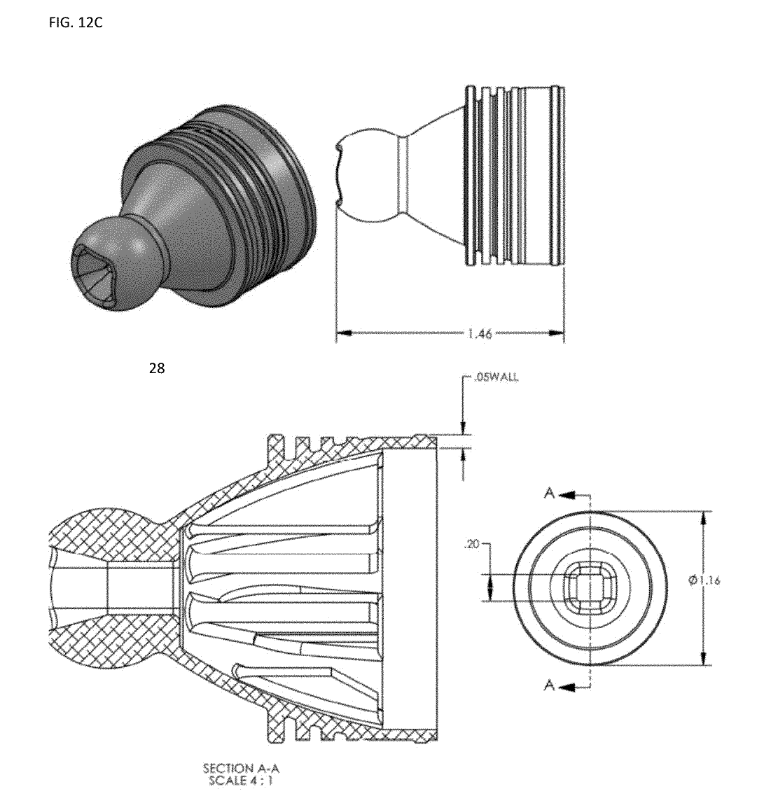

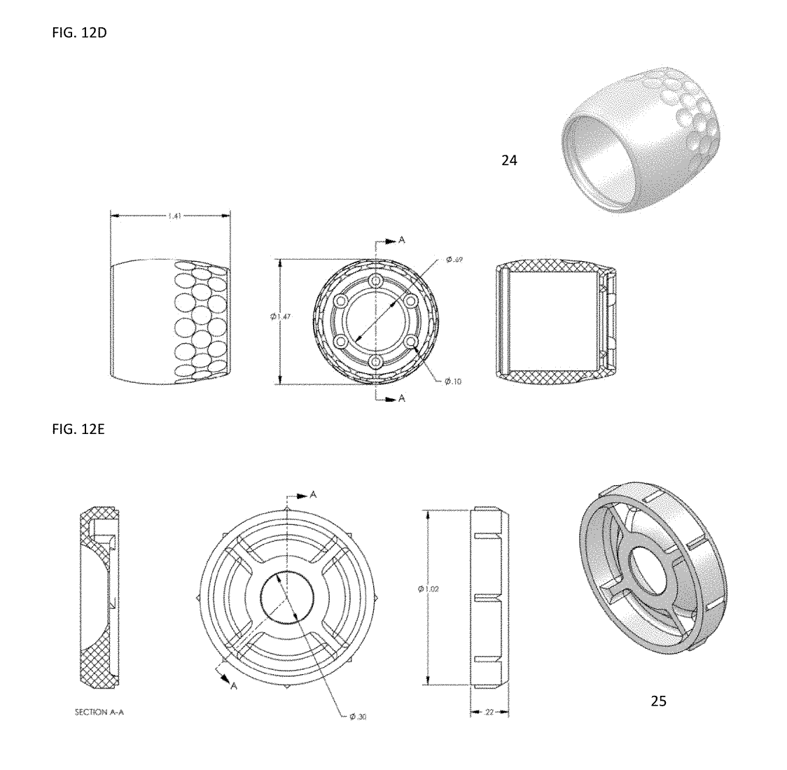

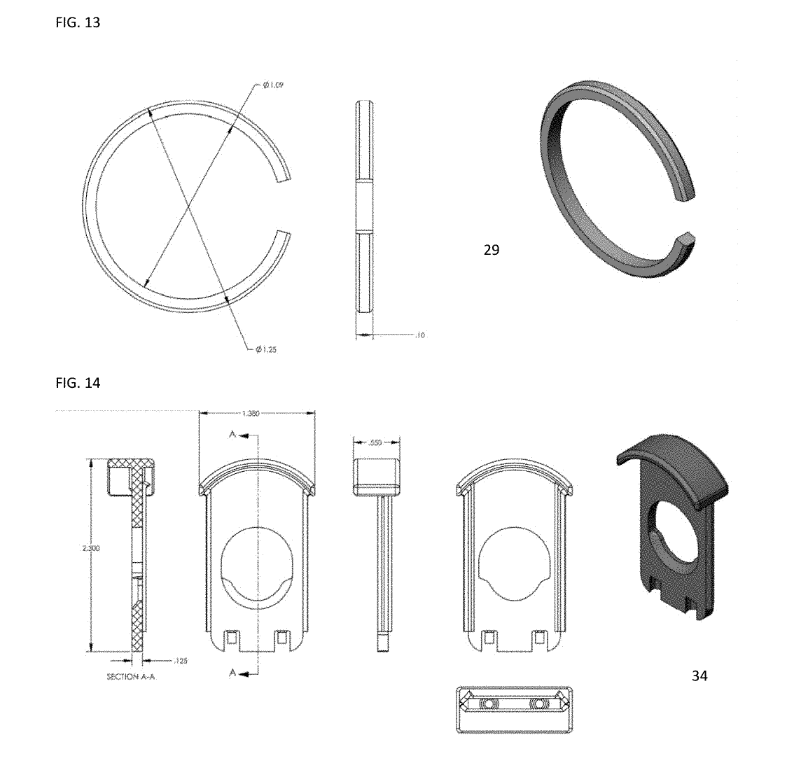

FIGS. 4, 5, 8, 12, and 13 show details of lamp 6. FIG. 4 shows details of the lamphead assembly. FIG. 4 shows lamphead aperture ring 24, lamphead lens retainer 23, lamphead lens 22, and lamphead screws 21. FIG. 5 shows further details of the lamphead assembly. FIG. 5 shows lamphead aperture ring 24, lamphead reflector 25, screw 26, lamphead PCB 27, lamphead ball joint body 28, and retainer 29. FIG. 5 further shows a line drawing and 3-dimensional rendering of assembled lamp 6. FIG. 8 shows a close up and dimensions of ball joint cap 17. FIG. 9 shows a close up and dimensions of balljoint socket 18. FIG. 12A shows a close up view of lamphead lens 22 and exemplary dimensions. FIG. 12B shows a close up view of lamphead lens retainer 23 and exemplary dimensions. FIG. 12C shows a close up view of lamphead lens ball joint 28 and exemplary dimensions. FIG. 12D shows a close up view of lamphead aperture ring 24 and exemplary dimensions. FIG. 12E shows a close up view of lamphead reflector 25 and exemplary dimensions. FIG. 13 shows a close up view of retainer 29 and exemplary dimensions.

The present disclosure is not limited to particular light source for use in light 1. In some embodiments, the light or lamp source comprises one or more light emitting diodes (LEDs), fluorescent, halogen, neon, incandescent, etc. In some embodiments, the light source emits light in the visible, ultraviolet, or infrared portions of the spectrum. In some embodiments, the light 1 comprises two or more different light sources that emit light of different wavelengths. In some embodiments, a switch or other activator is provided that permits a user to switch between different light options. For example, in some embodiments, a white light is used for a portion of a medical procedure and a second light (e.g., UV) is used for a second portion of a medical procedure to illuminate a contrast agent, dye, or other component contained in a patient tissue (e.g., to identify surgical margins; e.g., for assessing tumor boundaries). The switch may be contained on the lighting device or may be remote (e.g., a foot switch) that sends a signal (e.g., via wireless communication) to the light to switch configurations. In some embodiments, the light is configured to pulse. For example, in some embodiments, light of one wavelength is provided in a first pulse and light of a second wavelength is provided in a second pulse.

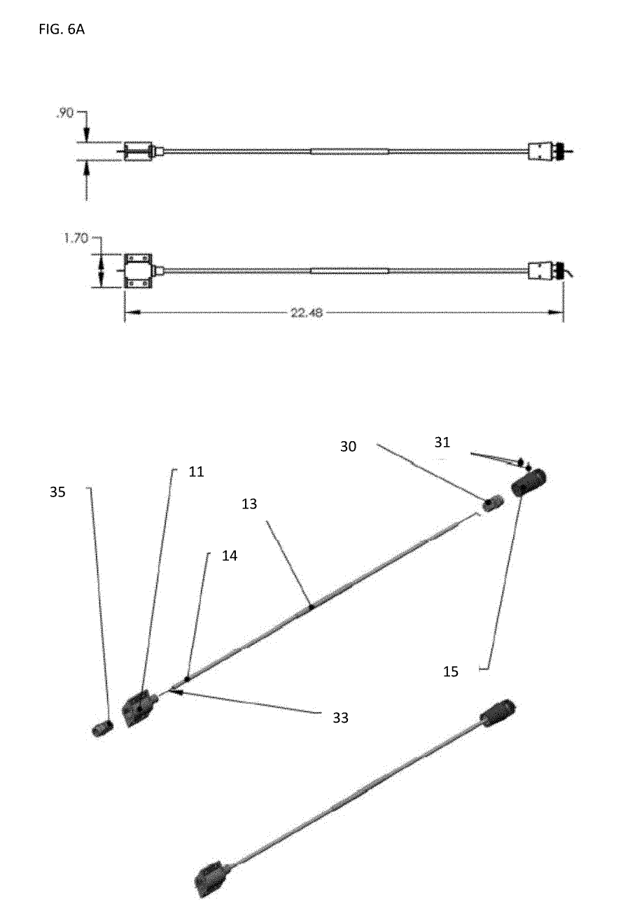

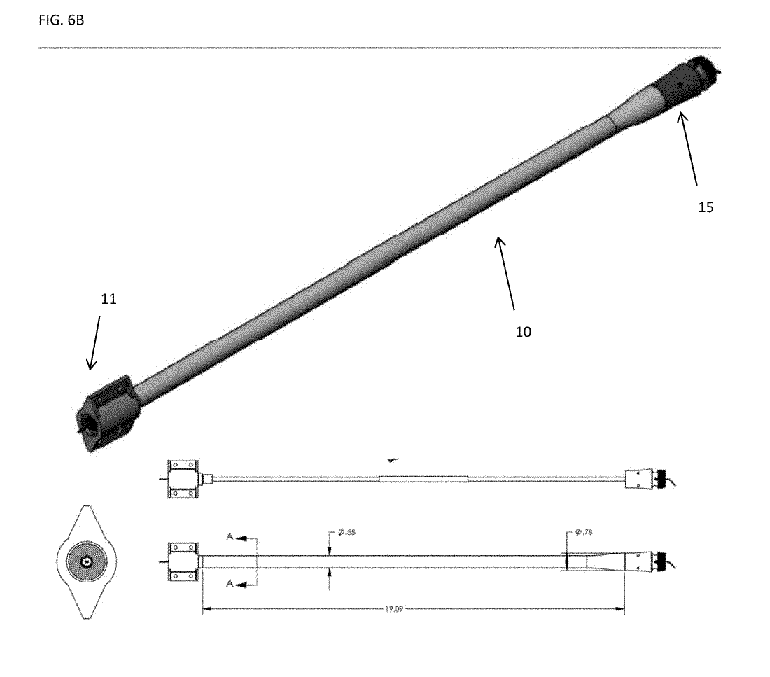

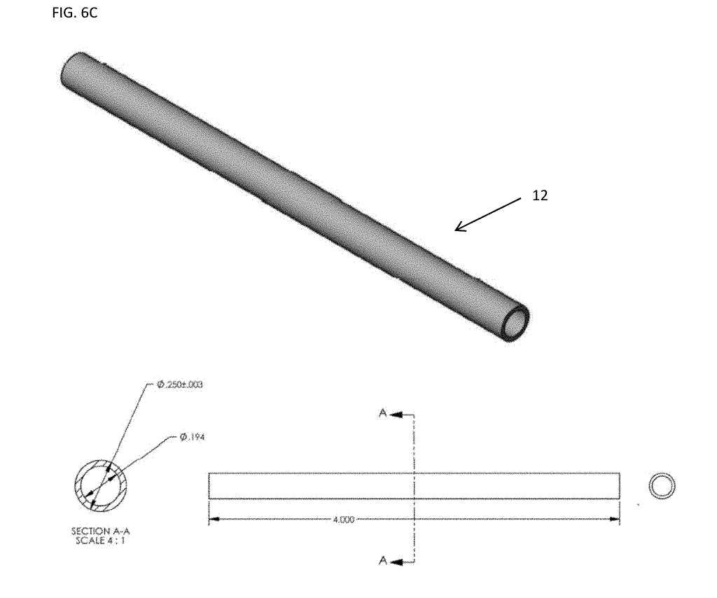

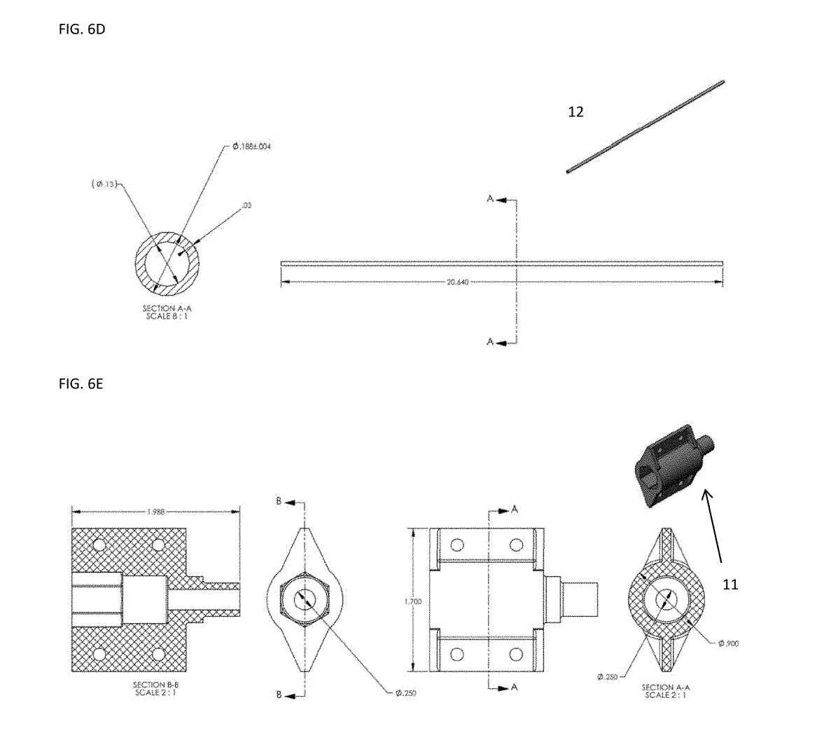

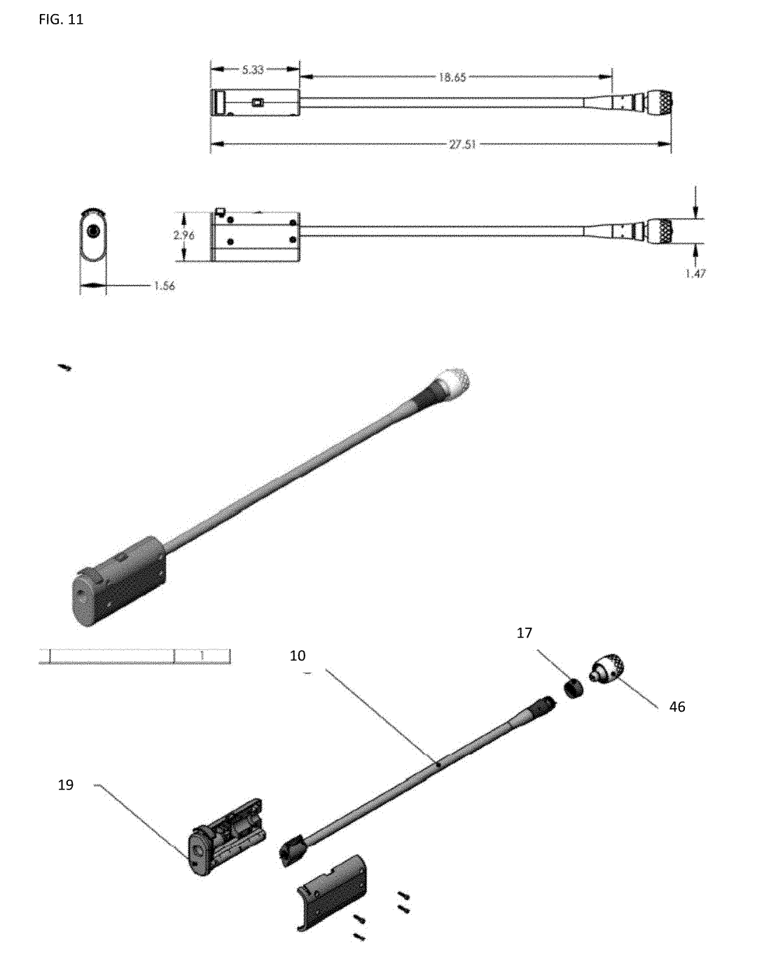

FIGS. 6 and 11 show a detail view of an exemplary flexible arm 10. Shown are flexible components 14 and rigid component 13. FIG. 6A further shows connector 35, battery housing 11, wire 33, connector 30, ball joint socket 15, and flexible arm screws 31. FIG. 6B shows a close up of the arm 10 showing battery housing 11 and ball joint socket 15. FIG. 6C shows a close up of tube 12. FIG. 6D shows a cross section and exemplary dimensions of tube 12. FIG. 6E shows a close up and exemplary dimensions of battery housing 11. FIG. 11 shows a close and dimensions of aspects of flexible arm 10. Shown are battery housing support end 19, ball joint cap 17, and lamphead assembly 46.

In some embodiments, the lengths of the flexible sections and the rigid section are present at a ratio of 1:2 to 2:1 or any values therein between (e.g., 1:1.9, 1:1.8, 1:1.7, 1:1.6, 1:1.5, 1:1.4, 1:1.3, 1:1.2, 1:1.1, 1:1, 1.1:1, 1.2:1, 1.3:1, 1.4:1, 1.5:1, 1.6:1, 1.7:1, 1.8:1, 1.9:1), although other ratios are specifically contemplated. In some embodiments, the length of the entire light including flexible and rigid components and power supply is 10-40 inches in length (e.g., 10, 15, 20, 25, 30, 35, or 40 inches or a fraction thereof). In some embodiments, the rigid component is 1-10 inches in length (e.g., 1, 2, 3, 4, 5, 6, 7, 8, 9, or 10 inches or a fraction thereof). In some embodiments, the flexible component(s) is 1-15 inches in length (e.g., 1, 2, 3, 4, 5, 6, 7, 8, 9, 10, 11, 12, 13, 14, or 15 inches or a fraction thereof).

In some embodiments, the flexible arm comprises power connector 11 configured for connection to a power component (e.g., battery pack) and lamp connector 15 configured for attached to a lamp component. In some embodiments, the flexible arm comprises a tube 12 that contains the rigid and flexible components.

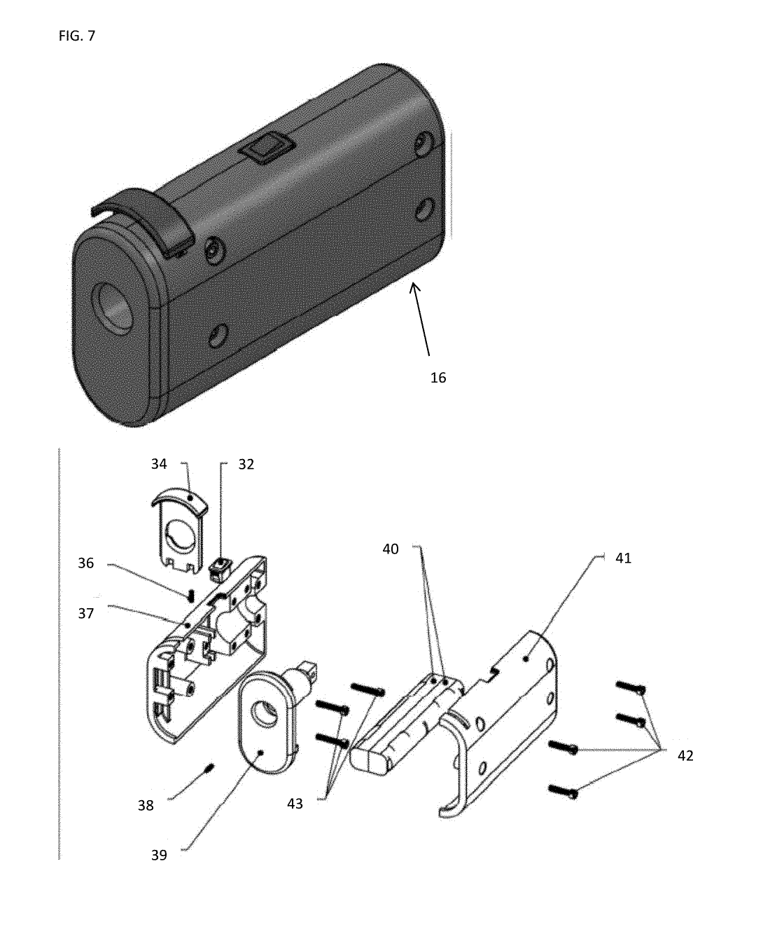

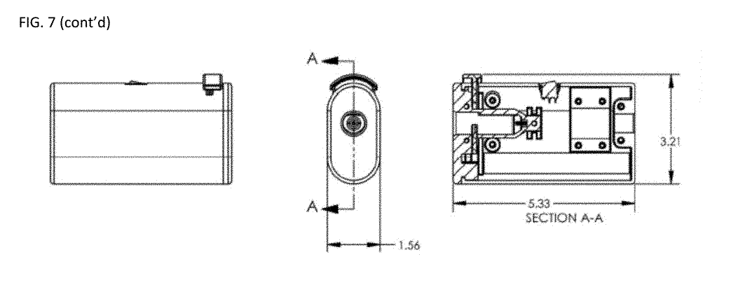

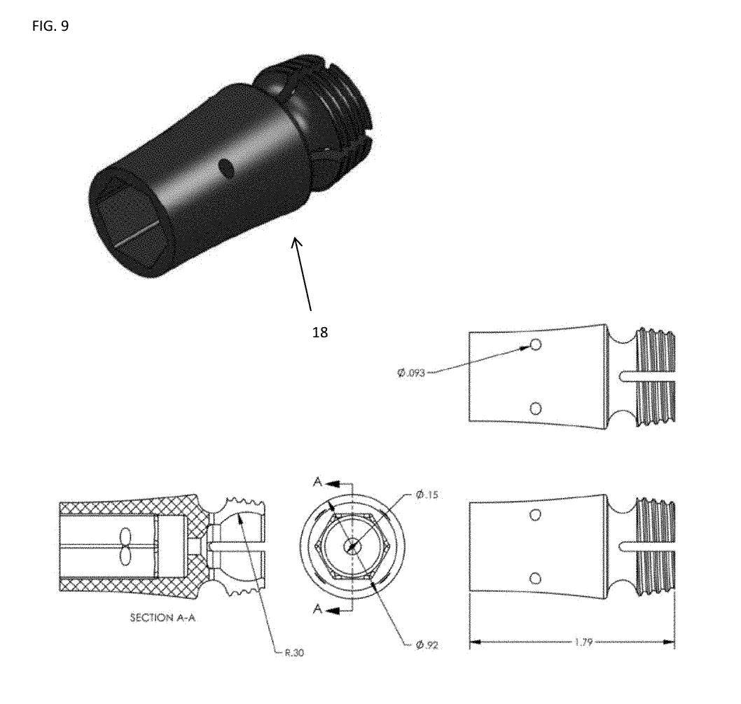

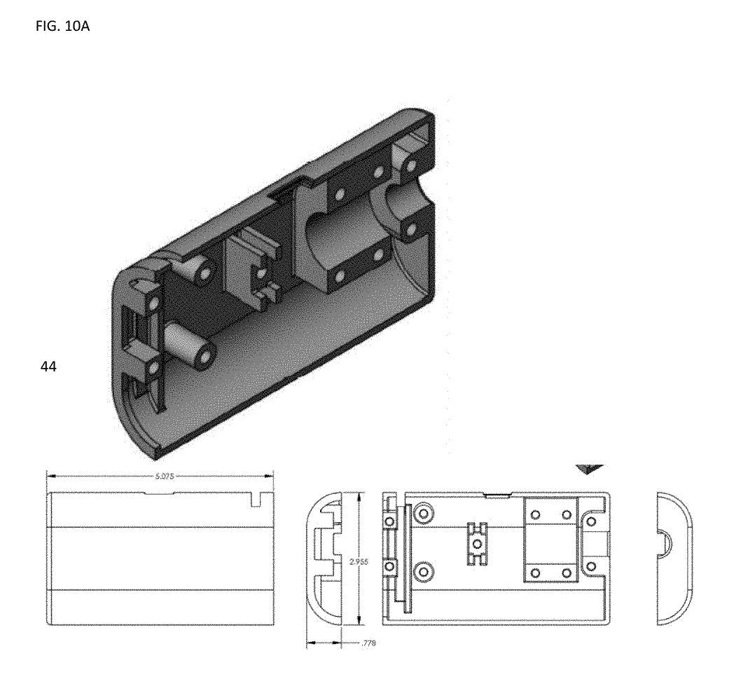

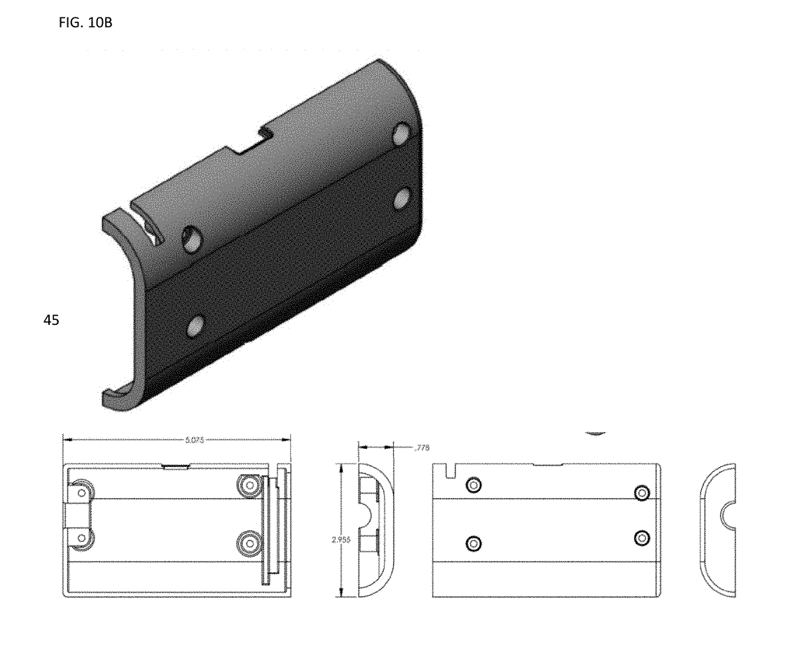

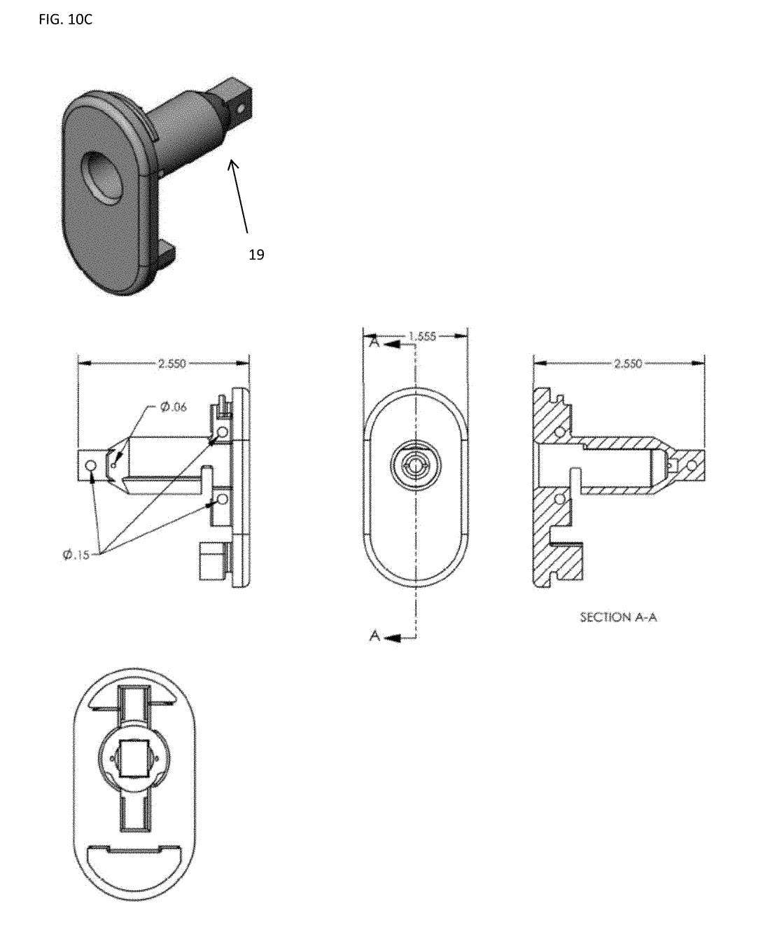

FIGS. 7, 10, and 14 show a detail view of an exemplary power supply (e.g., battery pack) 16. FIG. 7 shows left battery housing 37, right battery housing 41, battery housing support end 39, slide lock 34, cartridge spring 38, screws 42 and 43, battery 40, and digital key 32. FIGS. 10A and 10B show close ups and dimensions of left battery housing 44 and right battery housing 45. FIG. 19C shows a close up and dimensions of battery housing support end 19. FIG. 14 shows a close up view of slide lock 34 and exemplary dimensions. In some embodiments, slide lock 34 is replaced with a twisting bayonet lock (not shown).

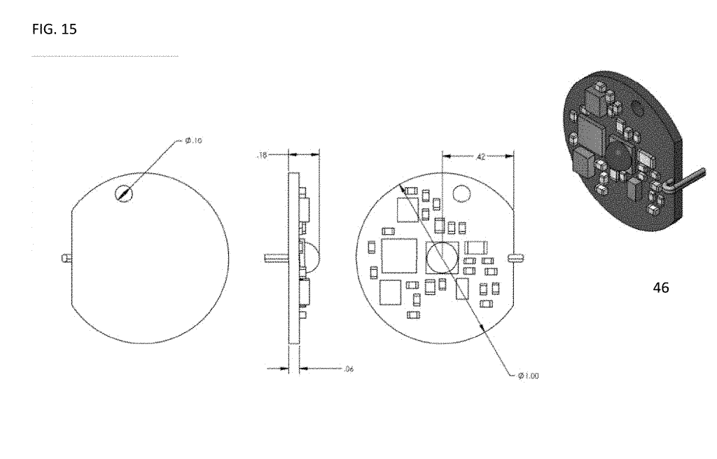

FIG. 15 shows a close up view of PCB board 46 and exemplary dimensions. Table 1 below shows exemplary sizes and sources for components of the device and system.

TABLE 1

| | | | | | Part | Wall | | Part |
|---|---|---|---|---|---|---|---|---|
| | | | Part | Part Size | Volume | Thickness | Projected | Weight |
| PART | Description | Process | Material | (In) | (In)\hat{ }3 | (In) | Area(In\hat{ }2) | (LBS) |
| Top Level | | Assembly | | | | | | |
| Assembly | | | | | | | | |
| Battery Hsg Asm | | Assembly | | | | | | |
| Cpt-A Battery | | Injection Molded | ABS-Black | 5.075 × 2.955 × 0.778 | 3.64 | 0.1 | 14.996 | 0.13 |
| Hsg Left | | | | | | | | |
| Cpt-A Battery | | Injection Molded | ABS-Black | 5.075 × 2.955 × 0.778 | 2.5367 | 0.1 | 14.996 | 0.0935 |
| Hsg Right | | | | | | | | |
| Cpt-A Battery | | Injection Molded | ABS-Black | 2.550 × 2.955 × 1.555 | 1.7234 | 0.25 | 40% | 0.0623 |
| Hsg SupportEnd | | | | | | | | |
| Cpt-A Slide Lock | | Injection Molded | Acetyl-Black | 2.300 × 1.380 × .550 | 0.31 | 0.125 | 90 | 0.011 |
| 99362A300 | McMaster- | | | | | | | |
| | Insert-Screw | | | | | | | |
| | in #6-32 | | | | | | | |
| Spring, Carriage, | | Std Part | | | | | | |
| CSC 10007 | | | | | | | | |
| 92196A150 | McMaster- | Std Part | 410 Stainless | | | | | |
| | Screw #6-32- | | Steel | | | | | |
| | SHCS × 0.75 | | | | | | | |
| 92196A151 | McMaster- | Std Part | 410 Stainless | | | | | |
| | Screw #6-32- | | Steel | | | | | |
| | SHCS × 0.625 | | | | | | | |
| InLine AA | | Assembly | | | | | | |
| Battery Asm | | | | | | | | |
| McM 7712K15 | McMaster- | | | | | | | |
| AA InLine | Battery | | | | | | | |
| BatteryHolder | Holder | | | | | | | |
| AA Battery | Battery | Std Part | | | | | | |
| DigKey-CH865- | Digikey | Std Part | | | | | | |
| ND | Switch | | | | | | | |
| Cpt-A FlexArm | | OverMold | Santoprene | 22.48 × 1.7 × .92 | 4.2197 | 0.24 | 10.726 | 0.1495 |
| Center OM | | | 201-80-Black | | | | | |
| Cpt-A FlexArm | | Assembly | | | | | | |
| Asm Insert | | | | | | | | |
| Cpt-A FlexArm | | Injection Molded | ABS-Black | 1.988 × 17 × .9 | 0.07468 | 0.162 | 2.58 | 0.0275 |
| End BatteryHsg | | | | | | | | |
| Cpt-A Flexarm- | Mc Master- | | Aluminum | | | | | |
| Tube | 5177K62- | | | | | | | |
| | 3/16 | | | | | | | |
| | Aluminum | | | | | | | |
| | Tubing | | | | | | | |
| 50915K214 | Mc Master- | | | | | | | |
| | Tube Fitting | | | | | | | |
| | 3/16 to | | | | | | | |
| | 1/8NPT | | | | | | | |
| Cpt-A BallJoint | | Injection Molded | | 1.79 × .92 × .92 | 0.445 | 0.18 | 1.48 | 0.0164 |
| Socket | | | | | | | | |
| 97395A433 | McMaster | | | | | | | |
| | Dowel Pin- | | | | | | | |
| | 3/32Dia × | | | | | | | |
| | 0.75 | | | | | | | |
| Cpt-A Flexarm- | McMaster- | Std Part | | | | | | |
| Support | 5176K23 - | | | | | | | |
| | 1/4 in steel | | | | | | | |
| | tubing | | | | | | | |
| Cpt-A FlexArm | Red and | Std Part | | | | | | |
| 2-Wire | Black 22 | | | | | | | |
| | gauge wire c | | | | | | | |
| | 30 inches | | | | | | | |
| LampHead Asm | | Assembly | | | | | | |
| Cpt-A LampHead | | Die Cast | Aluminum | 1.12 × 1.12 × 1.41 | 0.3441 | 0.05 | 0.9852 | 0.0336 |
| BallJoint Body | | | | | | | | |
| Light Head | | Assembly | | | | | | |
| Aperture SubAsm | | | | | | | | |
| Cpt-A LampHead | | Injection Molded | ABS-White | 1.47 × 1.47 × 1.41 | 0.711 | 0.157 | 1.7 | 0.0259 |
| Aperture Ring | | | | | | | | |
| Cpt-A LampHead | | Die cast | Aluminum | 1.15 × 1.15 × .1 | 0.0556 | 0.1 | 1.039 | 0.0054 |
| Lens Retainer | | | | | | | | |
| Cpt-A LampHead | Henglilens- | Standard Part | Lexan | | | | | |
| Lens | HL-19.8T | | | | | | | |
| 92210A017 | McMaster #1- | Std Part | Stainless | | | | | |
| | 72 FHCS × | | Steel | | | | | |
| | 0.1875 | | | | | | | |
| Cpt-A LampHead | | Injection Molded | ABS- | 1.06 × 1.06 × .222 | 0.0705 | 0.05 | 0.817 | 0.00261 |
| Reflector | | | HiTemp | | | | | |
| SMT 1 Inch PCB- | | Assembly | SMT | | | | | |
| 0556-00 | | | | | | | | |
| 90272A077 | McMaster #2- | Std Part | 18-8 | | | | | |
| | 56 × 0.25 | | Stainless | | | | | |
| | 18-8 Stainless | | | | | | | |
| Cpt-A Retainer | | Injection Molded | ABS-Black | 1.25 × 1.25 × .1 | 0.026 | 0.1 | 0.2695 | 0.001 |
| Cpt-A BallJoint | | Injection Molded | ABS-Black | 1.12 × 1.12 × 0.550 | 0.1609 | 0.179 | 0.9852 | 0.0059 |
| Cap | | | | | | | | |

In some embodiments, the light is provided in a system with the solid support 2 and clamp 3. In some embodiments, solid support 2 (FIG. 3) is a rigid metal or other material (e.g. rigid plastic or resin). In some embodiments, the solid support is a single straight tube or rod or other shape. In some embodiments, the solid support comprises one or more segments. In some embodiments, one or more of the segments is rigid or flexible. In some embodiments, one or more of the segments are straight or curved or another geometry. In some embodiments, the solid support 2 is shaped and dimensioned to position the light 1 in an ideal location within the surgical field, while eliminating or minimizing interference by either the light 1 or solid support 2 with the surgeon or other medical personnel or equipment.

In some embodiments, the solid support is sterilizable (e.g., via autoclave). In some embodiments, the solid support attaches to a medical procedure surface. In some embodiments, the solid support is attaches to the medical procedure surface using clamp 3. In some embodiments, the medical procedure surface is a surgical bed (e.g., bed rail). In some embodiments, the height of the solid support is adjustable (e.g., via the clamp). For example, in some embodiments, the solid support slides through the clamp until the desired height is reached. The clamp is then tightened and the solid support is locked in place at the desired height.

In some embodiments, systems comprise one or more software components (e.g. located on the solid support or lighting components, remotely, or a combination).

In some embodiments, cameras and or microphones provided with the system survey the surgical environment and record useful information and/or provide useful feedback to the surgeon, the patient, or others. For example, in some embodiments, video and or sound information collected is sent to and evaluated by a remote user (or computer) and provides feedback to improve the procedure or to evaluate the procedure for after-procedure feedback. In some embodiments, the system monitors, via video or audio, the treating physician to identify signs of fatigue and provides a warning (e.g., alarm light or sound) to notify the physician that they should remain alert or take some other action (e.g., drink coffee, swap shifts with another physician, etc.).

In some embodiments, one or more motors are provided with the system that control movement or positioning of one or more components of the system. In some embodiments, a motor is located at the lamp and positions the direction of the lamp. In some embodiments, a motor is located in the arm portion of the lighting component, at the juncture between the lighting component and the support arm, and/or at the junction of the support arm and the table or table rail. In some embodiments, the motors are remotely controlled to control the position of one or more portions of the system. In some embodiments, the motors are controlled by a foot pedal, joystick, or other external device. In some embodiments, the system comprises a body-tracking feature that detects an object in the room and moves the light in response to movement of the object. The object may be the physician's head (e.g., eyes) (e.g., identified by video capture, a fiducial located on the physician, etc.), arms, or hands or a medical device or a location on a patient. In some preferred embodiments, head tracking is employed such that the light moves to simulate lighting that would have been provided by a headlight worn by the physician. In some embodiments, system components are automatically moved to adjust for lighting quality, avoidance of shadows at the surgical site, or other desired optimizations. In some embodiments, remote components of the system communicate wirelessly, for example, using WiFi, Bluetooth, near-field communication technology, or other protocols.

The devices and systems described herein find use in a variety of medical procedures. The lights find use in any medical procedure that utilizes a sterile field and detailed lighting. In some embodiments, the medical procedure is surgery, for example transplant surgery, although other surgeries and medical procedures are specifically contemplated.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

What is claimed is:

1. A disposable, sterile, surgical light, comprising:
a lamp operably linked to a multi-segment flexible arm comprising at least one rigid section and at least one flexible section.

2. The light of claim 1, wherein said flexible arm comprises two flexible sections disposed on either side of a single rigid section.

3. The light of claim 2, wherein the lengths of said flexible sections and said rigid section are present at a ratio of 1:2 to 2:1.

4. The light of claim 1, wherein said light further comprises a power source.

5. The light of claim 4, wherein said power source is a battery.

6. The light of claim 1, wherein said light further comprises a support attachment component.

7. The light of claim 6, wherein said support attachment component is a component of said power source.

8. The light of claim 1, wherein said light further comprises a camera.

9. The light of claim 8, wherein said camera is attached to said flexible arm via a camera attachment clip.

10. The light of claim 1, wherein said lamp is focusable.

11. The light of claim 1, wherein said light further comprise an on-off switch.

12. The light of claim 11, wherein said on-off switch is located on said power supply.

13. The light of claim 1, wherein said lamp comprises at least one light source.

14. The light of claim 13, wherein said lamp comprises at least two light sources, wherein each of said light sources emits light of a different wavelength or range of wavelengths.

15. The light of claim 13, wherein said light source is configured to emit pulses of light.

16. The light of claim 14, wherein said light source is configured to switch between said two light sources.

17. A system, comprising:
a) at least one light of claim 1; and
b) a solid support configured to attach to said light using said support attachment component.

18. The system of claim 17, wherein said solid support attaches to a medical procedure surface.

19. The system of claim 17, wherein said solid support is attached to said medical procedure surface using a clamp.

20. A method of lighting a medical procedure, comprising:
a) contacting the system of claim 17 with a medical procedure surface; and
b) illuminating said medical procedure surface with said light.
